(19) United States
(12) Patent Application Publication
(10) Pub. No.: US 2014/0363418 A1
(43) Pub. Date: Dec. 11, 2014

(54) METHOD OF PRODUCING COLLAGEN FROM HYDROLYZED EGG MEMBRANE

(71) Applicant: MOLECULAR BIOLOGY INTERNATIONAL, INC., Sioux Center, IA (US)

(72) Inventor: Robert den Hoed, Sioux Center, IA (US)

(21) Appl. No.: 13/913,870

(22) Filed: Jun. 10, 2013

(51) Int. Cl. C07K 14/78; A61K 38/47; A61K 31/7008; A61K 31/728; A61K 31/726; A61K 31/737; A61K 38/39; A61K 38/40

(52) U.S. Cl. CPC C07K 14/78; A61K 38/39; A61K 38/47; A61K 38/40; A61K 31/728; A61K 31/726; A61K 31/737; A61K 31/7008
USPC 424/94.61; 435/273

(57) ABSTRACT

A method of producing collagen from hydrolyzed egg membrane includes combining 95% alcohol, cold water, a bacterial neutralase and/or alcalase, 14L, sodium bisulfite, and egg membrane. Once combined the solution is mixed slowly and then heated to a desired pH range. Once a desired temperature is reached, the heated solution is set aside to digest for a prolonged period of time. Next, the digested solution is centrifuged and filtered. Finally, the filtered solution is spray dried and packaged.

### BACKGROUND OF THE INVENTION

This invention is directed to a method of producing collagen from hydrolyzed egg membranes and more particularly to producing collagen from hydrolyzed egg membrane having lysozyme, ova transferrin, and sialic acid (LOS).

Eggshell membranes are well known as a source of valuable bioactive materials, including collagen, that have widespread applications in medical, health and cosmetic industries for joint and wound healing and skin care. To date, a major drawback to their use has been the difficulty in solubilizing these materials in a sufficiently stable and active pure form at an industrial scale so that high yield is achieved in an economic manner without using caustic solvents.

The amount of protein solubilized from the starting material by known processes is low, the techniques are not cost-effective, and often the recovered protein components do not maintain their native activity. Therefore an inexpensive process for producing collagen from hydrolyzed egg membranes while maintaining both yield, purity and activity of the solubilized protein is needed, particularly one suited for commercial scale implementation.

An objective of the present invention is to provide a method of solubilization of egg membranes under neutral conditions without use of caustic solvents.

A further objective of the present invention is to produce an improved collagen through a method that uses hydrolyzed egg membranes.

These and other objectives will be apparent to one of ordinary skill in the art based upon the following written description, drawings, and claims.

### SUMMARY OF THE INVENTION

A method of producing collagen from hydrolyzed egg membrane includes combining 95% alcohol, cold water, a bacterial neutralase and/or alcalase, 14L, sodium bisulfite, and egg membrane. Once combined the solution is mixed slowly and then heated to a desired pH range. Once a desired temperature is reached, the heated solution is set aside to digest for a prolonged period of time. Next, the digested solution is centrifuged and filtered. Finally, the filtered solution is spray dried and packaged.

### BRIEF DESCRIPTION OF THE FIGURES

FIG. 1 is a schematic diagram of the environment of performing a method of producing collagen from hydrolyzed egg membranes; and

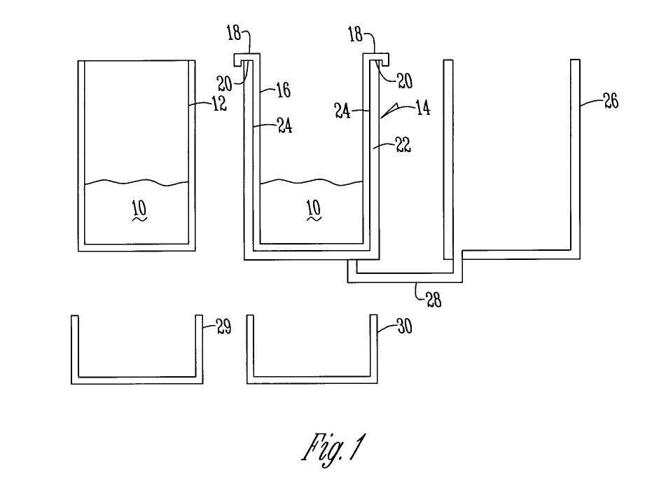

FIG. 2 is a flow diagram of a method of producing collagen from hydrolyzed egg membranes.

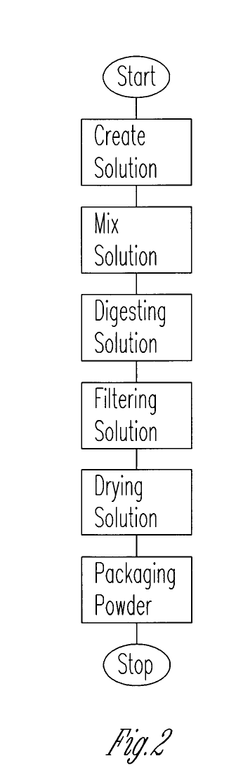

### DETAILED DESCRIPTION OF THE PREFERRED EMBODIMENTS

A method of making collagen from a hydrolyzed egg membrane includes the step of creating a solution 10 by combining 95% alcohol, cold water, a bacterial neutralase and/or alcalase, 14L, sodium bisulfite, and dried or moist egg membrane. In a preferred embodiment the combined solution includes 750 ml of 95% alcohol, the addition of cold water to a 15 liter mark, 90 mls of alcalase 660L, 30 mls of 14L, 36 grams of sodium bisulfate, and 1500 grams of egg membrane. Once combined the solution 10 is mixed slowly and heated to 60 degrees C. such that the solution 10 reaches a pH level between 4 and 10 pH. Preferably the solution 10 is mixed in a jacketed tank 12 so that the solution 10 can be heated.

Once the temperature of the solution 10 reaches 60 degrees C. the solution 10 is set aside and left alone in order to permit the solution 10 to digest for a period of between two to forty-eight hours. After digesting, the solution 10 is added to a centrifuge device 14 where the solution 10 is centrifuged at 2000 rpm for five minutes. The centrifuge device 14 has a filter 16 with a lip 18 that fits over the top edge 20 of a drum 22 such that the filter 16 is spaced from the interior wall 24 of the drum 22. Preferably the filter 16 is made of a nylon poly material having 1-25 microns. When centrifuged particles are maintained on the interior of the filter 16, while the filtered solution is pressed outwardly toward the interior wall 24 of the drum 22 and then pumped to a holding tank 26 through a discharge tube 28. From the holding tank 26, the filtered solution is pumped to a sprayer 29 where the filtered solution is spray dried. The resulting collagen powder is then transferred to a drum 30 for packaging and distribution.

The dried filtered solution which results in a collagen powder has a minimum of 20% protein, 1.0% calcium, 2% ova transferrin, 2% lysozyme, 0.1% sialic acid, 0.1% hyaluronic acid, 1% mucopolysaccharide, and 0.1% chondroitin. In addition, the collagen powder preferably includes a minimum of 31.23% wt of mucopolysaccharide, a minimum of 35.4% wt chondrotin sulfate sodium, a minimum of 5.985% wt of hyaluronic acid, and a minimum of 5.337% wt of glucosamine₆, Further, preferred is a collagen powder having a molecular weight of 10 to 200 Kda. Thus, a method of solubilazation of egg membrane under neutral conditioning without caustic solvents has been disclosed that, at the very least, meats all the stated objectives.

What is claimed:

1. A method of producing collagen from hydrolyzed egg membranes, comprising the steps of:
creating a solution by combining 95% alcohol, cold water, at least one of a bacterial neutralase and alcalase, 14L, sodium bisulfite, and egg membrane;
mixing and heating the combined solution to a temperature of 60 degrees C.;
digesting the solution for a period of between 2 and 48 hours;
filtering the solution through a centrifuge device; and
spraying the solution dry.

2. The method of claim 1 wherein the combined solution includes 750 mls of 95% alcohol, cold water added to a 15 liter mark, 90 mls of alcalase 660L, 30 mls of 14L, 36 grams of sodium bisulfite, and 1500 grams of egg membrane.

3. The method of claim 1 wherein the heated solution has a pH level of between 4 and 10 pH.

4. A collagen powder produced by the method of claim 1 that has a minimum of 20% protein, 1.0% calcium, 2% ova transferrin, 2% lysozyme, 0.1% sialic acid, 0.1% hyaluronic acid, 0.1% mucopolysaccharide, and 0.1% chondroitin.

5. A collagen powder produced by the method of claim 1 having a minimum of 31.23% wt of mucopolysaccharide, a minimum of 35.4% wt chondrotin sulfate sodium, a minimum of 5.985% wt of hyaluronic acid, and a minimum of 5.337% wt of glucosamine₆.

6. A collagen powder produced by the method of claim 1 having a molecular weight between 10 and 200 Kda.
